(19) United States
(12) Patent Application Publication
(10) Pub. No.: US 2023/0277076 A1
(43) Pub. Date: Sep. 7, 2023

(54) BLOOD FLOW IN RENAL VASCULATURE USING ELECTRICAL RESISTANCE AND ASSOCIATED SYSTEMS, DEVICES, AND METHODS

(71) Applicant: KONINKLIJKE PHILIPS N.V., EINDHOVEN (NL)

(72) Inventor: James David CEZO, COLORADO SPRINGS, CO (US)

(21) Appl. No.: 18/098,364

(22) Filed: Jan. 18, 2023

Related U.S. Application Data
(60) Provisional application No. 63/302,456, filed on Jan. 24, 2022.

(51) Int. Cl. A61B 5/027; A61B 5/00

(52) U.S. Cl. CPC A61B 5/027; A61B 5/742; A61B 5/6876

(57) ABSTRACT

A system includes an endovascular catheter or guidewire that is positioned within a blood vessel. The catheter or guidewire includes two pairs of electrodes. One pair of electrodes is in communication with a power source. The other pair of electrodes is in communication with an amplifier and/or rectifier. The system also includes a processor circuit configured for communication with the endovascular catheter or guidewire. The processor circuit controls one pair of electrodes to provide a constant electrical current from one electrode to another. The processor circuit controls the other pair of electrodes to measure a voltage difference. The processor circuit then determines, based on the voltage difference, a blood flow metric of the blood within the blood vessel and outputs the blood flow metric to a display.

### CROSS-REFERENCE TO RELATED APPLICATIONS

This application claims priority to and the benefit of U.S. Provisional Application No. 63/302,456, filed Jan. 24, 2022, which is incorporated by reference herein in its entirety.

### TECHNICAL FIELD

The present disclosure relates generally to measuring blood flow. In particular, a pair of electrodes emits electrical energy within a renal artery of a patient and an additional pair of electrodes measures electrical potential to determine blood flow within the renal artery.

### BACKGROUND

Physicians use many different medical diagnostic systems and tools to monitor a patient’s health and diagnose medical conditions. In particular, blood flow is an important metric used by physicians to assess the condition of a blood vessel in a patient. In addition, blood flow at various locations within a patient’s vasculature can be an indicator or symptom of broader medical conditions. In that regard, it is important for a physician to be able to measure blood flow. In addition, different patients may exhibit different responses to various methods or devices or measuring blood flow. As a result, a variety of methods and corresponding devices may be needed to fit the particular medical conditions and requirements of individual patients.

In addition, blood flow is a metric which may be measured to determine whether a renal denervation procedure would be beneficial to a patient experiencing hyper-tension as well as whether a previously performed renal denervation procedure was successful or needs to be performed again. Renal denervation involves ablating or otherwise disabling the nerves of the renal artery. Because the renal nerves cause the renal artery to expand or contract in response to various stimuli, the renal nerves may be a cause of unnecessary high blood pressure in a patient. By disabling these nerves, blood pressure may be decreased. However, renal denervation is not an effective treatment in all patients or at all locations within the renal vasculature of a patient. It is often difficult for a physician to determine whether a renal denervation will effectively address hypertension for a patient as results of renal denervation are highly patient-specific. As a result, a physician may perform a renal denervation procedure without success. This may be because the patient was not a patient which would respond positively to a renal denervation procedure or because the renal denervation procedure was performed in an incorrect region of the renal vasculature. Performing a renal denervation procedure with little to no effect on the patient unnecessarily subjects a patient to a traumatic and time-consuming procedure and wastes costly resources.

### SUMMARY

Embodiments of the present disclosure are systems, devices, and methods for measuring blood flow in the renal vasculature of a patient using electrical resistance. Aspects of the present disclosure advantageously provide a physician with an additional method of measuring blood flow within a patient. In addition, aspects of the present disclosure may be used to determine whether a patient would be an appropriate candidate for a renal denervation procedure and whether a renal denervation procedure performed previously was effective.

In some aspects, an endovascular device may be positioned within the renal artery of a patient. The endovascular device includes at least four electrodes. One pair of electrodes is positioned at a distal location on the device and another pair of electrodes is positioned at a proximal location along the device. The outermost electrodes (e.g., the most distal electrode and the most proximal electrode) receive electrical energy from a power source. These electrodes emit electrical energy into the blood within the renal artery of the patient. As this electrical energy is emitted, the blood between the outermost electrodes completes a circuit and electrical energy may flow through the blood. As the outermost electrodes emit electrical energy as described, the remaining electrodes between the outermost electrodes measure electrical potential. For example, the electrical energy within the renal artery may result in a graduated difference in electrical potential within the blood between the outermost electrodes. In some aspects, an electrical resistance between the inner electrodes may be measured. As blood flow increases within the renal artery, the measured electrical resistance may decrease. Similarly, as blood flow within the renal artery decreases, the measurement electrical resistance may increase. A processor circuit receives the measurements from the innermost electrodes and may determine a blood flow within the renal artery based on the measurements.

In an exemplary aspect, a system is provided. The system includes an endovascular catheter or guidewire configured to be positioned within a blood vessel of a patient, wherein the endovascular catheter of guidewire comprises: a first set of electrodes comprising a first electrode and a second electrode; and a second set of electrodes comprising a third electrode and a fourth electrode; and a processor circuit configured for communication with the endovascular catheter or guidewire, wherein the processor circuit is configured to: control the first set of electrodes such that an electrical current is transmitted between the first electrode and the second electrode; control the second set of electrodes to obtain a first measurement of a voltage difference between the third electrode and the fourth electrode; determine, based on the first measurement of the voltage difference, a first flow metric for blood flow within the blood vessel; and output the first flow metric to a display in communication with the processor circuit. The first flow metric may be a first value of the flow metric.

In one aspect, a first electrical circuit is formed between the first electrode and the second electrode with the blood flow, and a second electrical circuit is formed between the third electrode and the fourth electrode with the blood flow. In one aspect, the system further includes a power source configured for electrical communication with the processor circuit and the first set of electrodes. In one aspect, to control the first set of electrodes, the processor circuit is configured to control the power source to provide the electrical current to only the first electrode. In one aspect, the first electrode is configured to transmit the electrical current to the second electrode using a first electrical circuit formed between the first electrode and the second electrode by the blood flow. In one aspect, the second set of electrodes is configured to obtain the first measurement of the voltage difference using a second electrical circuit formed between the third electrode and the fourth electrode by the blood flow. In one aspect, the electrical current comprises a constant electrical current. In one aspect, the system includes at least one of an amplifier or a rectifier configured for electrical communication with the second set of electrodes. In one aspect, the processor circuit is configured to control at least one of the amplifier or the rectifier to determine the first measurement of the voltage difference based on the electrical communication with the third electrode and the fourth electrode. In one aspect, the third electrode and the fourth electrode are positioned between the first electrode and the second electrode. In one aspect, the first electrode and the second electrode comprise a first spacing, and the first electrode and the third electrode comprise a second spacing less than the first spacing. In one aspect, the blood vessel comprises a renal artery. In one aspect, the endovascular catheter or guidewire is further configured to control the second set of electrodes to obtain a second measurement of a voltage difference between the third electrode and the fourth electrode; and determine, based on the first measurement of the voltage difference, a second flow metric for the blood flow. In one aspect, the processor circuit is configured to output the second flow metric to the display. In one aspect, the processor circuit is configured to compare the first flow metric and the second flow metric. In one aspect, the processor circuit is configured to provide an output based on the comparison to the display. In one aspect, the blood vessel is a renal artery, the first measurement is obtained before a stimulation of a sympathetic nervous system of the patient and the second measurement is obtained after the stimulation, and the processor circuit is configured to: determine whether renal denervation is recommended for the patient based on the comparison; and provide an output based on the determination to the display. In one aspect, the blood vessel is a renal artery, the first measurement is obtained before a renal denervation procedure and the second measurement is obtained after the renal denervation procedure, and the processor circuit is configured to: determine if the renal denervation procedure was successful based on the comparison, and provide an output based on the determination to the display. In one aspect, a first electrical circuit is formed between the first electrode and the second electrode with the blood flow; a second electrical circuit is formed between the third electrode and the fourth electrode with the blood flow; and the processor circuit is further configured to maintain a constant electrical flow of the first electrical circuit.

In an exemplary aspect, a system is provided. The system includes an endovascular catheter or guidewire configured to be positioned within a renal artery of a patient, wherein the endovascular catheter of guidewire comprises: an outer set of electrodes comprising a first electrode and a second electrode; and an inner set of electrodes comprising a third electrode, and a fourth electrode; and a processor circuit configured for communication with a power source and the endovascular catheter or guidewire, wherein the processor circuit is configured to: control the power source to provide constant electrical current to the first electrode such that the constant electrical current is transmitted from the first electrode to the second electrode with a first electrical circuit formed between the first electrode and the second electrode by blood flow within the renal artery; control the third electrode and the fourth electrode to obtain a measurement of a voltage difference with a second electrical circuit formed between the third electrode and the fourth electrode with the blood flow, wherein the voltage difference is generated based on a change in the blood flow; determine, based on the measurement of the voltage difference, a flow metric representative of the change in the blood flow; and provide an output based on the flow metric to a display in communication with the processor circuit.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of a data acquisition and carotid bodies stimulation system 100, according to aspects of the present disclosure.

FIG. 2 is a schematic diagram of a processor circuit, according to aspects of the present disclosure.

FIG. 3 is a diagrammatic view of an endovascular device positioned within a renal anatomy, according to aspects of the present disclosure.

FIG. 4 is a diagrammatic view of an endovascular device positioned within a renal anatomy, according to aspects of the present disclosure.

FIG. 5 is a diagrammatic view of an endovascular device positioned within a branch of the renal anatomy, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Aspects of the present disclosure may include various principles taught in of U.S. Application No. 18/086,511, filed Dec. 21, 2022, which is incorporated by reference herein in its entirety.

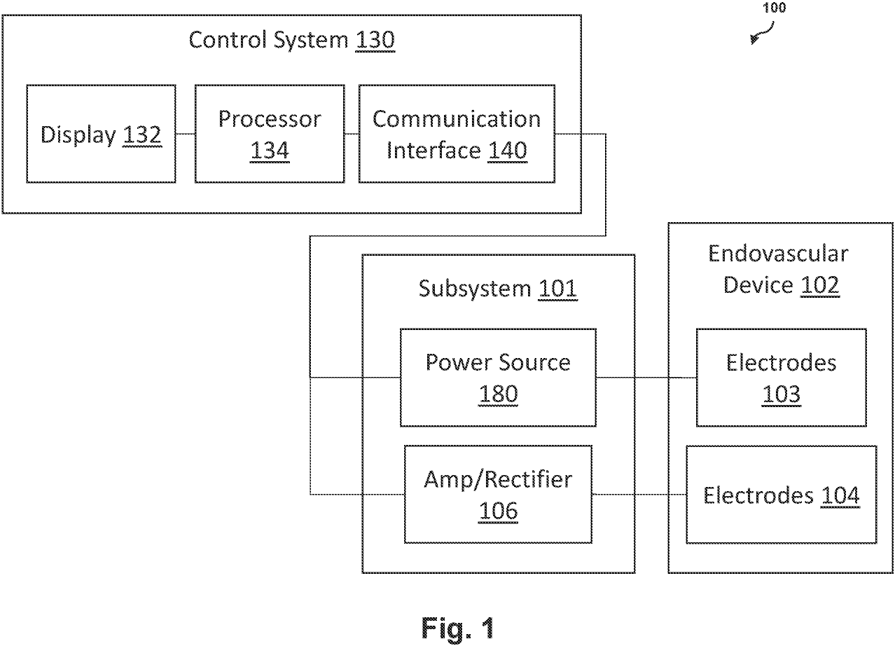

FIG. 1 is a schematic diagram of a data acquisition and carotid bodies stimulation system 100, according to aspects of the present disclosure. In some embodiments, and as shown in FIG. 1, the system 100 may include a control system 130, one or more subsystems, and one or more endovascular devices, such as the endovascular device 102.

The system 100 shown in FIG. 1 may advantageously assist a physician in measuring the flow of blood within a vessel of a patient. In some embodiments, the system 100 may assist a physician in measuring the blood flow within a renal artery of a patient.

The control system 130 may be configured to generate various commands to control subsystems, such as the subsystem 101. The control system 130 may be additionally configured to generate commands to control various devices. For example, the control system 130 may be configured to generate commands to control the endovascular device 102. In some embodiments, the control system 130 may be configured to generate command signals to control one or more electrodes, such as a set of electrodes 103 and/or a set of electrodes 104. The subsystem 101 may alternatively be referred to as a control panel that allows for reading and interpretation of sensor data as well as control of elements in various embodiments.

The control system 130 may be any suitable device or system. For example, the control system 130 may include a user input device, a processor circuit 134, a communication interface 140, and/or a display 132. The control system 130 may include additional devices, components, or elements. In some embodiments, the control system 130 may be a computer, such as a laptop, a tablet device, or any other suitable computational device. In some embodiments, the control system 130 may include additional elements related to communication between the control system 130, or the processor circuit 134 of the control system 130, and other systems, subsystems, or devices. For example, the control system 130 may include an interface module. In some examples, the control system 130 may include a patient interface module (PIM).

In some embodiments, the control system 130 may additionally be configured to receive various data from other systems, subsystems, or devices. For example, the control system may be configured to receive data related to blood pressure, blood flow, or the velocity of blood within a vessel of a patient. The control system may receive blood flow data from a flow sensor and/or electrodes of the device 102 as will be described in more detail hereafter.

A user input device in communication with the control system 130 may be any suitable device. For example, the user input device may be configured to receive a user input via one or more buttons or mouse clicks. The user input device may additionally be configured to receive a user input via any other method. For example, the user input device may receive a user input via a touch on a touch screen, an auditory input such as speech or other sounds. In some embodiments, the user input device may be a keyboard, a mouse, a touch screen, one or more buttons, a microphone, or any other suitable device configured to receive inputs from a user.

The processor circuit 134 may be configured to generate, receive, and/or process any various data. For example, the processor circuit 134 may be in communication with the memory storage system of the control system 130. The processor circuit 134 may be configured to execute computer readable instructions stored on the memory storage system of the control system 130. The processor circuit 134 may additionally be configured to generate outputs based on any suitable computer readable instructions the processor circuit 134 may execute. For example, the processor circuit 134 may generate an output configured to be received by elements of the device 102 to begin to emit or measure electrical energy. For example, the processor circuit 134 may generate an output to be received by the device 102, instructing the set of electrodes 103 to begin to emit electrical energy into the surrounding environment, such as blood within a renal artery. In some embodiments, the processor circuit 134 may be further configured to process data received from the devices with which the control system 130 is in communication. For example, the processor circuit 134 may process electrical potential data. The processor circuit 134 may additionally process other data. In some embodiments, the processor circuit 134 may be configured to generate one or more graphical user interfaces to be output to a display, such as the display 132. In some embodiments, the processor circuit 134 may be additionally configured to receive user inputs from a user input device, such as the user input device described previously.

The display 132 may be any suitable display. The display 132 may also be any suitable device. For example, the display 132 may include one or more pixels configured to display regions of an image to a user of the system 100. The display 132 may be in communication with the processor circuit 134 of the control system 130. In this way, the display 132 may receive instructions and/or images to display to a user of the system 100. In some embodiments, the display 132 may show a user a view of the data received and/or processed by the processor circuit 134. The display 132 may additionally convey various recommended actions or prompts for the user of the system 100 from the processor circuit 134. In some embodiments, the display 132 may additionally or alternatively be a user input device. For example, the user of the system 100 may select various elements within a graphic shown on the display 132 to direct the processor circuit 134 of the control system 130 to perform various actions or commands.

The subsystem 101 may be in communication with the processor circuit 134, as shown in FIG. 1. The subsystem 101 may be any suitable device, system, or subsystem. For example, the subsystem 101 may be configured to receive commands from the processor circuit 134 of the control system 130 and send these commands or signals to one or more devices, such as the endovascular device 102. In some embodiments, the subsystem 101 may process signals received from the processor circuit 134 and/or the endovascular device 102. In this way, the subsystem 101 may facilitate communication between the processor circuit 134 and a device, such as the endovascular device 102. In some embodiments, the subsystem 101 may be configured to control the electrodes 103. For example, a power source 180 of the subsystem 101 may provide electrical energy to the electrodes 103. The power source 180 may also be referred to as a current source, a voltage source, an electrical energy source, or an electrical power source. This electrical energy may be emitted by the electrodes 103 into the surrounding environment, such as blood within a renal artery of a patient. In this way, the subsystem 101 and the electrodes 103 may together form an electrical stimulation system.

The subsystem 101 may also be configured to control one or more electrodes 104 of the endovascular device 102. In some embodiments, the endovascular device 102 may include one or more electrodes 104. In some embodiments, and as shown in FIG. 1, the endovascular device 102 may include the electrodes 103 and the electrodes 104. The electrodes 104 may be configured to measure electrical potential differences between two or more electrodes 104. This data may be transmitted to the amplifier/rectifier 106 of the subsystem 101. In this way, the endovascular device 102 may be configured to both emit electrical energy via the electrodes 103 and measure electrical potential differences via the electrodes 104. The subsystem 101 may be configured to receive command signals from the processor circuit 134. For example, in response to a user input from the user of the system 100, or in response to other computer readable instructions, the processor circuit 134 may generate a command for the subsystem 101 to begin to receive electrical potential difference data. In such an embodiment, the subsystem 101 may receive such a command from the processor circuit 134 and may measure one or more electrical potential differences or electrical signals and transmit this data to the processor 134. Similarly, the processor circuit 134 may transmit a command to the subsystem 101 to stop acquiring data

As shown in FIG. 1, the endovascular device 102 may be a single device configured to perform multiple functions. For example, the endovascular device 102 may transmit electrical energy and may measure electrical potential differences. However, in some embodiments, the electrodes 103 may be housed on a separate device from the electrodes 104. In that regard, one device including the set of electrodes 103 may be positioned within a renal artery of a patient or at any other location within a patient vasculature. With this device positioned within a renal artery, a second device including the set of electrodes 104 may be positioned at the same location or a different location within the patient vasculature.

As shown in FIG. 1, the power source 180 and the amplifier/rectifier 106 of the subsystem 101 may be housed within the same subsystem 101. However, these elements may be alternatively housed within separate subsystems.

In some embodiments, the electrodes 103 and/or 104 of the endovascular device 102 may be configured to contact or be positioned proximate to a vessel luminal wall. In some embodiments, the electrodes 103 and/or 104 may be positioned within a central portion of a vessel. The electrodes 103 may emit an electrical pulse or a constant electrical voltage. The electrical field created by the electrodes 103 may cause electrical energy to pass from one electrode 103 to another electrode 103 causing a change in voltage between each of these electrodes 103. The electrodes 104 may be configured to measure this difference in voltage between the electrodes 104 based on the electrical potential or voltage of the surrounding medium (e.g., blood). Additional aspects of the electrodes 103 and/or 104 will be described in more detail hereafter.

In some aspects, any of the systems, devices, sensors, methods, principles, or any teachings of the present invention may be substantially similar to the teachings of U.S. Provisional Application No. 63/300,536, filed Jan. 18, 2022, which is incorporated by reference herein in its entirety.

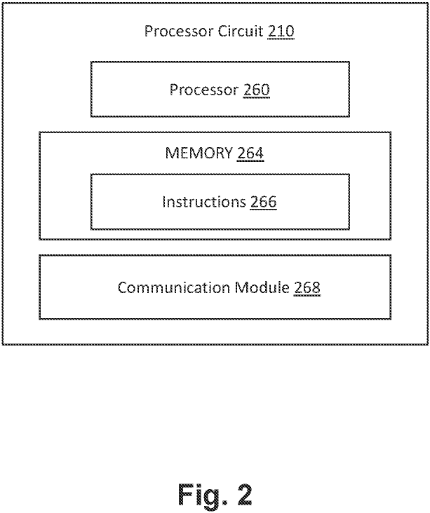

FIG. 2 is a schematic diagram of a processor circuit, according to aspects of the present disclosure. The processor circuit 210 may be implemented in the control system 130 (e.g., as shown in FIG. 1), or any other suitable location. In an example, the processor circuit 210 may be in communication with any of the devices, systems, or subsystems described in the present disclosure. For example, the processor circuit 210 may be in communication with a blood flow sensing device, a pressure sensing device, an extraluminal imaging device, a nerve stimulation device, a nerve ablation device or any other device, system, or subsystem. The processor circuit 210 may include a processor 134 and/or a communication interface. One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 260, cause the processor 260 to perform the operations described herein with reference to any of the devices, system, or subsystems described. Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 210, the devices, systems, or subsystems described herein, the display 132, processor circuit 134, or communication interface 140 (FIG. 1). In that regard, the communication module 268 can be an input/output (I/O) device. In some instances, the communication module 268 facilitates direct or indirect communication between various elements of the processor circuit 210 and/or various described endovascular or extraluminal devices, systems, and/or the host 130 (FIG. 1).

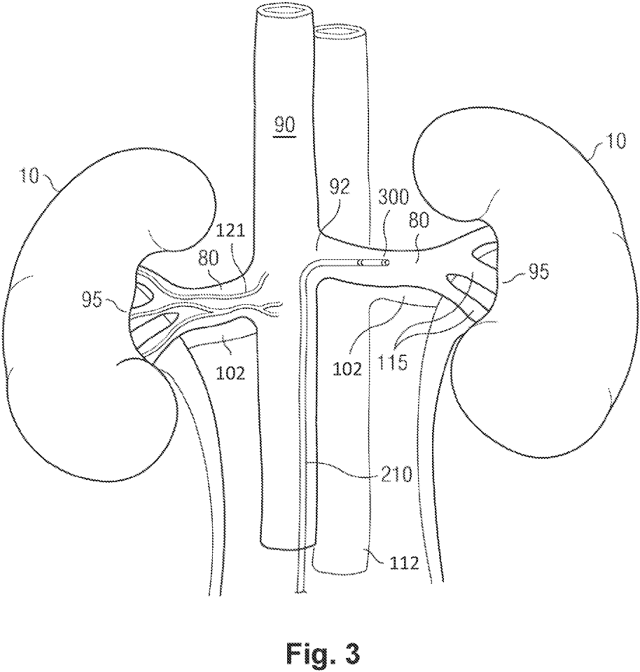

FIG. 3 is a diagrammatic view of an endovascular device positioned within a renal anatomy, according to aspects of the present disclosure. FIG. 3 illustrates an intravascular device 210 disposed within the human renal anatomy. The human renal anatomy includes kidneys 10 that are supplied with oxygenated blood by right and left renal arteries 80, which branch off an abdominal aorta 90 at the renal ostia 92 to enter the hilum 95 of the kidney 10. The abdominal aorta 90 connects the renal arteries 80 to the heart (not shown). Deoxygenated blood flows from the kidneys 10 to the heart via renal veins 102 and an inferior vena cava 112. Specifically, a flexible elongate member of the intravascular device 210 is shown extending through the abdominal aorta and into the left renal artery 80. In alternate embodiments, the intravascular device 210 may be sized and configured to travel through the inferior renal vessels 115 as well. Specifically, the intravascular device 210 is shown extending through the abdominal aorta and into the left renal artery 80. In alternate embodiments, the catheter may be sized and configured to travel through the inferior renal vessels 115 as well.

Left and right renal plexi or nerves 121 surround the left and right renal arteries 80, respectively. Anatomically, the renal nerve 121 forms one or more plexi within the adventitial tissue surrounding the renal artery 80. For the purpose of this disclosure, the renal nerve is defined as any individual nerve or plexus of nerves and ganglia that conducts a nerve signal to and/or from the kidney 10 and is anatomically located on the surface of the renal artery 80, parts of the abdominal aorta 90 where the renal artery 80 branches off the aorta 90, and/or on inferior branches of the renal artery 80. Nerve fibers contributing to the plexi arise from the celiac ganglion, the lowest splanchnic nerve, the corticorenal ganglion, and the aortic plexus. The renal nerves 121 extend in intimate association with the respective renal arteries into the substance of the respective kidneys 10. The nerves are distributed with branches of the renal artery to vessels of the kidney 10, the glomeruli, and the tubules. Each renal nerve 121 generally enters each respective kidney 10 in the area of the hilum 95 of the kidney, but may enter the kidney 10 in any location, including the location where the renal artery 80, or a branch of the renal artery 80, enters the kidney 10.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 121, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 121. Renal denervation may help alleviate the symptoms and sequelae of hypertension by blocking or suppressing the efferent and afferent sympathetic activity of the kidneys 10.

In some embodiments, the vessel 80 is a renal vessel and the pulse wave velocity is determined in the renal artery. The processing system (e.g., the processor circuit 210) may determine various physiological parameters, such as the blood pressure, blood flow, blood flow velocity, pulse wave velocity (PWV), strain or constriction of the vessel, voltage measurements of renal nerves, or any other parameters in the renal artery. The processing circuit 210 may determine a renal denervation therapy recommendation based on these parameters in a renal artery. For example, patients that are more likely or less likely to benefit therapeutically from renal denervation may be selected based on the parameters measured. In that regard, based on these parameters measured corresponding to the renal vessel, the processing circuit 210 can perform patient stratification for renal denervation.

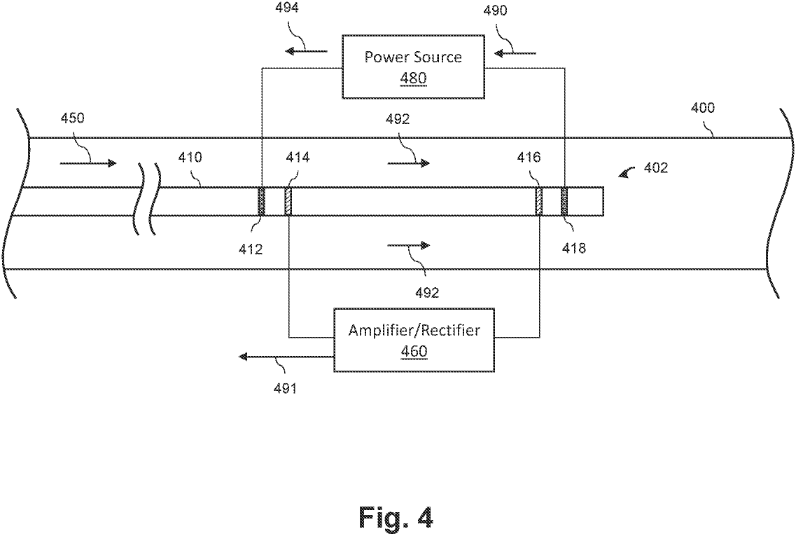

FIG. 4 is a diagrammatic view of an endovascular device positioned within a renal anatomy, according to aspects of the present disclosure. The device 402 may be one embodiment of the device 102 described with reference to FIG. 1. As shown in FIG. 4, the device 402 may be configured to be positioned within a blood vessel 400 of a patient. For example, as shown in FIG. 4, a diagrammatic view of a blood vessel 400 is provided. The blood vessel 400 may be a renal artery of the patient.

The device 402 may include a flexible elongate member 410, a proximal emitting electrode 412, a proximal measurement electrode 414, a distal measurement electrode 416, and a distal emitting electrode 418.

The flexible elongate member 410 may be sized and shaped, structurally arranged, and/or otherwise configured to be positioned within a body lumen 400 of a patient. The flexible elongate member 410 may be a part of guidewire and/or a catheter (e.g., an inner member and/or an outer member). The flexible elongate member 410 may be constructed of any suitable flexible material. For example, the flexible elongate member 410 may be constructed of a polymer material including polyethylene, polypropylene, polystyrene, or other suitable materials that offer flexibility, resistance to corrosion, and lack of conductivity. In some embodiments, the flexible elongate member 410 may define a lumen for other components to pass through. The flexible elongate member 410 may be sufficiently flexible to successfully maneuver various turns or geometries within the vasculature of a patient. The flexible elongate member 410 may be of any suitable length or shape and may have any suitable characteristics or properties.

As shown in FIG. 4, the proximal emitting electrode 412 and the distal emitting electrode 418 may be in communication with a power source 480. The power source 480 may be substantially similar to the power source 180 described with reference to FIG. 1. For example, the power source 480 may be configured to generate electrical energy at the electrodes 412 and/or 418. In that regard, a first electrical signal (such as an electrical current and/or an electrical voltage) may be generated via the electrodes 412 and 418. In some aspects, the first electrical signal is an electrical current (e.g., a constant electrical current). In some aspects, the first electrical signal(s) includes a first voltage at the first electrode and/or a second voltage at the second electrode, thereby creating an electrical current (e.g., a constant electrical current) between the first electrode and the second electrode. The power source 480 may include electrical circuitry configured to generate and output the first electrical signal to the electrode 412 and/or the electrode 418. The electrical circuitry can be a current generating/outputting circuit or a voltage generating/outputting circuit.

In one example, a constant current circuit of the power source 480 generates and outputs a constant current to the first electrode or the second electrode (e.g., only one of the first electrode or the second electrode). For example, the processor circuit controls the power source to provide the constant electrical current to the first electrode. Blood flow provides a portion of the electrical circuit between the first and second electrodes that carries the constant current from the first electrode to the second electrode.

In one example, a voltage circuit of the power source 480 generates and outputs electrical signal such that the first electrode comprises first voltage and second electrode comprises second voltage. Voltage circuit is configured to provide same or different voltage values at the first electrode and the second electrodes, that create/induce/generate an electrical current (e.g., a constant electrical current) between the first electrode and the second electrode. Blood flow provides a portion of the electrical circuit between the first and second electrodes that carries the constant current from the first electrode to the second electrode. In some aspects, whether electrical current is provided to the electrode 412 or the electrode 418 may depend on the direction of blood flow relative to the electrodes 412 and 418. For example, if blood is flowing in the direction shown in FIG. 4, the power source 480 may output the constant electrical current to the electrode 412. If blood is flowing in the opposite direction relative to the electrodes 412 and 418, the power source 480 may output the constant electrical current to the electrode 418 or vice versa. The power source 480 can be a direct current (DC) source in some aspects. The power source 480 can be a voltage source, in some aspects, configured to generate a DC signal between the first and second electrodes.

For example, the power source 480 may include a positive terminal. In some embodiments, the proximal emitting electrode 412 may be in electrical communication with a positive terminal of the power source 480. Similarly, the distal emitting electrode 418 may be in electrical communication with a negative terminal of the power source 480. In that regard, the processor circuit (such as the processor 134 and/or processor circuit 210) may control the power source 480 to output an electrical signal, such as a current and/or a voltage, to provide one voltage at the electrode 412 and a different voltage at the electrode 418. In some embodiments, the processor circuit may be configured to control the power source 480 to establish an electrical circuit of constant electrical current (i.e., constant amperage) formed of at least electrodes 412 and 418 and the blood moving between the electrodes 412 and 418. In that regard, an electrical circuit may be formed including the power source 480, the electrode 418, the electrode 412, the connecting conductors between these components and blood within the renal artery 400. This circuit may be illustrated as electrical energy passing from the electrode 418 to the negative terminal of the power source 480 as shown by the arrow 490, from the power source 480 to the electrode 412 as illustrated by the arrow 494, and from the electrode 412 to the electrode 418 as illustrated by the arrows 492 in FIG. 4.

In some aspects, a portion of the first electrical circuit (e.g., comprising electrodes 412, 418 and power source 480) and a portion of the second electrical circuit (e.g., comprising electrodes 414, 416 and amplifier/rectifier 460) physically and/or electrically overlap. In that regard, the portions that overlap are located within the blood vessel 400. The region of the blood vessel 400 where the portions of the two electrical circuits overlap is where blood flow measurements including blood flow changes may be calculated. For example, in this overlapped region, the electrical current is a known provided current, electrical voltage is measured, and, as a result, the electrical resistance is calculated. The region of the blood vessel 400 where the portions of the two electrical circuits overlap may be the region inside the blood vessel where the electrodes 412, 414, 416, 418 are located.

The electrodes 414 and 416 may be used to measure differences in voltage or electrical potential. For example, as shown in FIG. 4, the distal electrode 416 may be in communication with an amplifier/rectifier 460. The proximal electrode 414 may also be in communication with the same amplifier/rectifier 460. In some embodiments, the electrodes 414 and 416 may be configured to measure a voltage at each of their respective locations within the vessel 400. In this way, the electrodes 414 and 416 may approximately measure the difference in voltage potential of the blood between the locations of the electrodes 412 and 418 as the electrical energy from the power source 480 passes along this length of the vessel 400. In some aspects, the arrangement of the electrodes 414 and 416 spatially between the electrodes 412 and 418 positions the electrodes 414 and 416 within or inside the electrical circuit formed between the electrode 412, the electrode 418, and the blood. In some aspects, a second electrical signal (e.g., an electrical current and/or an electrical voltage) may be generated via the electrodes 414 and 416. In some aspects, the second electrical signal is an electrical voltage. In some aspects, blood flow may be calculated based on the provided electrical signals (e.g., constant electrical current provided by the electrodes 412 and 418 and voltage measured by the electrodes 414 and 416) because the current is known (supplied by the power source 480 via the electrodes 412 and 418) and the voltage is known (as measured by the electrodes 414 and 416). According to Ohm’s law (V = IR), the resistance may be calculated, which corresponds to blood flow. In some aspects, reference to electrical resistance may also be referred to as electrical impedance. In some aspects, the processor circuit can determine values of blood flow metric(s) from the calculated electrical resistance using a look up table or any other suitable relationship (accessible to the processor, stored in memory) between blood flow and electrical resistance.

In some aspects, a distance or spacing between the electrodes 414 and 416 may be less than a distance or spacing between the electrodes 412 and 418. In some aspects, the electrodes 412 and 418 may form a first set of electrodes and form a first electrical circuit in communication with the power source 480 and the electrodes 414 and 416 may form a second set of electrodes and form a second electrical circuit in communication with the amplifier and/or rectifier 460. In some aspects, the electrodes 414 and 418 may form a first set of electrodes and a first electrical circuit in communication with the power source 480 and the electrodes 412 and 416 may form a second set of electrodes and a second electrical circuit in communication with the amplifier and/or rectifier 460. In some aspects, the electrodes 412 and 416 may form a first set of electrodes and a first electrical circuit in communication with the power source 480 and the electrodes 414 and 418 may form a second set of electrodes and a second electrical circuit in communication with the amplifier and/or rectifier 460. In some aspects, the electrodes 412 and 414 may form a first set of electrodes and a first electrical circuit in communication with the power source 480 and the electrodes 416 and 418 may form a second set of electrodes and a second electrical circuit in communication with the amplifier and/or rectifier 460. In some aspects, the electrodes 416 and 418 may form a first set of electrodes and a first electrical circuit in communication with the power source 480 and the electrodes 412 and 414 may form a second set of electrodes a second electrical circuit in communication with the amplifier and/or rectifier 460.

The amplifier/rectifier 460 may be configured to amplify and rectify the potential measurements received from the electrodes 414 and 416. In some embodiments, the rectifier portion of the device 460 may be configured to convert the signals received from the electrodes 416 and 418 from an AC to a DC current. In other embodiments, the rectifier 460 may convert signals from a DC to an AC current. In some embodiments, the amplifier portion of the device 460 may be configured to amplify the signals received from the electrodes 414 and 416. For example, the amplifier may receive a signal or signals from the electrodes 414 and 416 which is on the order of microvolts. The amplifier may increase the amplitude of these signals to signals on the order of millivolts or volts. In some aspects, the subsystem 101 may include both an amplifier and a rectifier. In some aspects, the amplifier and rectifier may be housed within the same device or in separate devices. In some aspects, the subsystem 101 may include an amplifier or a rectifier.

In some embodiments, the amplifier/rectifier 460 may send various data 491 to a processor circuit, such as the processor circuit 210 described with reference to FIG. 2, or the processor 134 of the control system 130. In some embodiments, the amplifier/rectifier 460 may send the data 491 to a component of the subsystem 101. The data 491 sent by the amplifier/rectifier 460 may include voltage data, potential data, impedance data, resistance data, or any other data obtained by the electrodes 414 and 416 either separately or in combination. In some embodiments, the amplifier/rectifier 460 may be configured to pre-process the data received by the electrodes 414 and 416 in any suitable way.

Based on the data 491, a processor circuit of the system 100, including for example, the processor 134 or the processor circuit 210, may determine a resistance measurement associated with the length between the electrodes 414 and 416. This resistance measurement may be reflective of the electrical resistance within the vessel 400. In some embodiments, this resistance measurement may be used to determine a blood flow metric associated with the flow of blood in the vessel 400. In some aspects, the blood flow metric may be a blood flow rate or velocity. In some aspects, the blood flow metric may be a blood flow volume. In some aspects, the blood flow metric may be a blood flow volume per unit time, a blood flow volume per unit area, or any other blood flow metric. For example, blood might flow through the vessel 400 in a direction shown by the arrow 450. As described previously with reference to the power source 480, electrical energy may flow from the proximal electrode 412 to the distal electrode 418 in a direction shown by the arrows 492. As shown in FIG. 4, the direction of flow of electrical energy (shown by the arrows 492) may be the same as the direction of blood flow (shown by the arrow 450). As a result, as blood flow increases, the measured resistance between the electrodes 414 and 416 may decrease. By contrast, as the blood flow decreases, the measured resistance between the electrodes 414 and 416 may increase. In some aspects, blood flow measurements calculated based on the voltage measurements obtained by the electrodes 414 and 416 may be representative of a change in blood flow. For example, a change in blood flow may be induced by stimulation of the sympathetic nervous system of a patient (e.g., to determine if a patient is a good candidate for denervation). In some examples, a change in blood flow may result from a renal denervation procedure (e.g., to determine if a renal denervation was successful).

In some aspects, the blood flow measurement system described herein may be used to calculate an absolute blood flow measurement or a relative blood flow measurement. For example, the voltage measurements obtained by the electrodes 414 and 416 may be used to calculate a blood flow measurement value, including any of those described herein (e.g., unit blood volume per unit time). In other examples, differences in blood flow as blood flow changes in a vessel may be proportional to changes in voltage as measured by the electrodes 414 and 416. In that regard, while the device 402 is positioned within a vessel, a first voltage difference may be obtained by the electrodes 414 and 416 and associated with a baseline blood flow. As the blood flow changes and different voltages are measured by the electrodes 414 and 416, the different blood flow may be quantified and/or displayed to a user as values compared to the baseline blood flow (e.g., as a percentage, as a difference in voltage, or any other arbitrary units of comparison).

In some embodiments, a relation between the measured resistance between the electrodes 414 and 416 and the blood flow may be established. As a result, the system 100 may calculate and display blood flow measurements based on the difference in potential between the electrodes 414 and 416. In some embodiments, changes in blood flow measurements may be illustrated or calculated based on changes in the resistance measurements between the electrodes 414 and 416.

It is noted that the power source 480 may be configured to provide electrical energy in a different way. For example, electrical energy may flow from the power source 480 to the electrode 418 in an opposite direction and travel in a proximal direction within the vessel 400 to the electrode 412. In such an embodiment, as the blood flow increases within the vessel 400, the resistance or impedance measured between the electrodes 414 and 416 may increase. By correlation, if the blood flow were to decrease within the vessel 400 the resistance between the electrodes 414 and 416 may decrease.

In some aspects, flow measurements obtained with the device 402 may be used in assessing the predicted effectiveness of a renal denervation procedure before it is performed and/or the success of renal denervation procedure after it is performed. In that regard, a method of assessing a renal denervation may include stimulating the sympathetic nervous system. The sympathetic nervous system may be stimulated in a variety of ways. For example, the sympathetic nervous system may be stimulated by applying pressure to the carotid bodies of the patient, by an endovascular device positioned within the renal artery of the patient, by an external device, such as a patch positioned over the carotid bodies of the patient, by an endovascular device positioned within a renal artery of the patient, or by any other way.

As the sympathetic nervous system is stimulated, for example, by any of the ways previously described, the device 402 may be used to monitor the sympathetic nervous system for a response to the stimulation. For example, the response of the sympathetic nervous system may be monitored by monitoring the blood flow within the renal artery by the device 402.

The processor circuit of the system may then analyze the sympathetic nervous system response to determine whether the patient will respond to a renal denervation procedure. In some aspects, analyzing the sympathetic nervous system response may include comparing blood flow measurements obtained by the device 402 collected while the sympathetic nervous system was stimulated with metrics collected while the sympathetic nervous system was not stimulated. In some aspects, when a difference between metrics collected under stimulation and metrics collected while not under stimulation is observed, this may indicate that a patient will respond to a renal denervation procedure. In some aspects, this difference between metrics under stimulation and metrics not under stimulation (sometimes referred to as metrics at rest) may be compared to a threshold. For example, if this difference between metrics exceeds a predetermined threshold, a processor circuit of the system 200 may determine that the patient is likely to respond to a renal denervation procedure.

After performing the comparison of metrics under stimulation and metrics at rest, the processor circuit may generate a recommendation for a renal denervation procedure. A renal denervation procedure may include any procedure in which nerves surrounding the renal artery of a patient are disabled. For example, in some procedures, an endovascular device is positioned within the renal artery of the patient. With the device within the renal artery, electrodes of the device may ablate nerves surrounding the renal artery. This ablation procedure may be performed at various locations along either or both renal arteries of the patient. After a renal denervation procedure, the device 402 may be positioned within the renal artery and again obtain blood flow measurements with the sympathetic nervous under stimulation and at rest to determine if the renal denervation procedure was successful. If successful, a renal denervation procedure reduces blood pressure within a patient.

In that regard, after additional blood flow measurements obtained while the sympathetic nervous system is stimulated and blood flow measurements obtained while the sympathetic nervous system is not stimulated, the processor circuit may analyze the sympathetic nervous system response. After a renal denervation procedure has been performed, a difference between metrics collected under stimulation and metrics at rest may indicate that the renal denervation procedure was not successful. This difference in response to stimulation may be due in part by the renal nerves still responding to the stimulation, meaning that they were not sufficiently disabled. In some aspects, when there is little to no difference between metrics under stimulation and metrics at rest, it may indicate that the renal denervation procedure was successful. As described previously, the comparison of metrics under stimulation and metrics at rest may include comparing these metrics to a threshold. In addition, differences between metrics collected after the renal denervation procedure may be compared to differences between metrics collected before the renal denervation procedure. Any difference between these two differences in metrics may be compared to a predetermined threshold as well to determine whether a renal denervation procedure was successful. In that regard, comparisons of blood flow metrics may be made with other blood flow metrics or with threshold values.

In some aspects, sympathetic response may additionally be indicated by a change in blood pressure within the patient vasculature and/or the renal artery. In that regard, in some aspects, the endovascular device may include a pressure sensor positioned at any location along the device 402. The pressure sensor may provide pressure measurements to the processor circuit of the system while the electrodes are used to measure blood flow. In some aspects, these two metrics may provide a physician with a more accurate view of the sympathetic response. For example, a physician may confirm the accuracy of one metric (blood pressure or flow), by comparing it to the other. In some aspects, a pressure sensor may be positioned on a separate endovascular device and may serve a similar purpose. In some aspects, the pressure measurements and flow measurements may be used to determine a fluid resistance metric.

The results of determining either the predicted effectiveness of a potential future renal denervation procedure or the degree of success of a completed renal denervation procedure may be displayed in any suitable way. For example, the processor circuit may be configured to output to a display an output based on the comparison. This may include a value of the difference between blood flow metrics. In other embodiments, a binary value, graphical element, such as a symbol, various colors or shapes, or any other graphical element, a graph, chart, table, or any other method of displaying data.

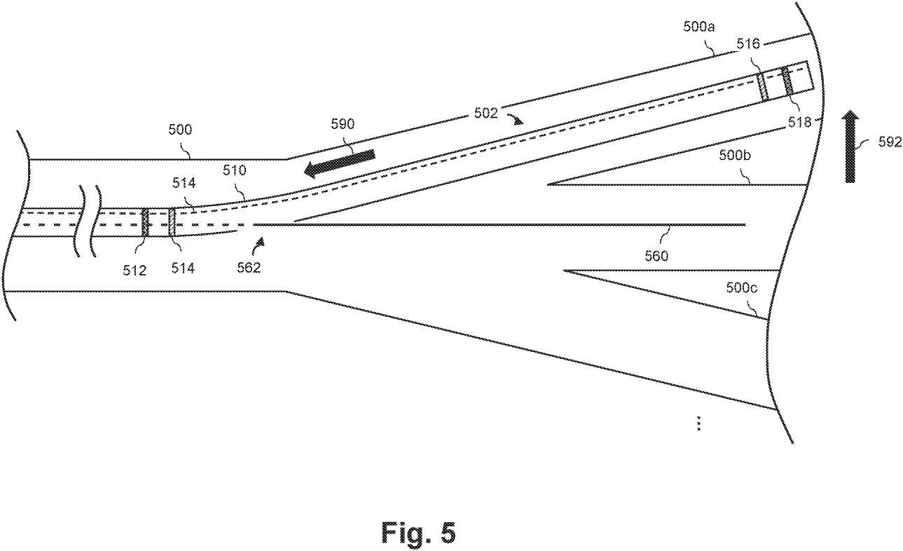

FIG. 5 is a diagrammatic view of an endovascular device positioned within a branch of the renal anatomy, according to aspects of the present disclosure. The device 502 may be one embodiment of the device 102 described with reference to FIG. 1. As shown in FIG. 5, the device 502 may be configured to be positioned within a blood vessel of a patient.

A renal artery 500 is shown in FIG. 5. The renal artery 500 may, at a distal end, split into multiple side branches. For example, a side branch 500a, a side branch 500b, and a side branch 500c are shown. It is noted that additional or fewer side branches may be included within the renal vasculature. The side branches 500a, 500b, and 500c may extend in a distal direction and terminate at a kidney. Following the renal artery 500 in a proximal direction, the renal artery 500 may join the aorta of the patient. While the device 502 is described with reference to the renal vasculature, as shown in FIG. 5, similar principles of deflection and device positioning may apply to any region of the patient vasculature.

In the embodiment shown, a portion of the device 502 may be positioned within one side branch (e.g., the side branch 500a) while a separate portion of the device 502 may be positioned within a different side branch (e.g., the side branch 500b). In some embodiments, the distal portion of the device 502 (e.g., a distal emitting electrode 518 and a distal measurement electrode 516) may be moved to different side branches within the renal vasculature without completely removing the device 502. The proximal portion of the device 502 (including a proximal emitting electrode 512 and a proximal measurement electrode 514) may be positioned within the renal artery 500 (e.g., not positioned within any of the side branches 500a, 500b, or 500c).

As shown in FIG. 5, a guidewire 560 may extend along a longitudinal lumen of the device 502. In some embodiments, the guidewire 560 may be positioned within the vasculature first. In the embodiment shown, the guidewire 560 may be positioned within the side branch 500b. The device 502 may then be positioned around the guidewire 560. For example, a lumen of the device 502 may be sized to receive the guidewire 560. At the opening 562, the device 502 may be positioned around the guidewire 560. The device 502 may then be moved along the guidewire through the patient vasculature to the renal vasculature. There, the device 502 may be positioned within the same side branch 500b with the guidewire 560. After measurements are made there, however, the device 502 may be moved in a proximal direction so as to exit the side branch 100b and return to the primary renal artery 500. There, the measurement portion of the device 502 may be deflected from the guidewire 560 so as to be positioned in a separate side branch (e.g., the side branch 500a) while the guidewire 560 remains in the same side branch (e.g., the side branch 500b).

In some embodiments, the device may include one or more pull wires 514. A pull wire (e.g., the pull wire 514) may be positioned within the device 502 or on an outer surface of the device 502. In some embodiments, the pull wire 514 may be attached to a side of the device 502 or a side of the flexible elongate member 510 of the device 502. In this way, when a physician, or other automated or robotic system, pulls on the pull wire 514, a force is exerted in the proximal direction shown by the arrow 590. Due to the flexible nature of the device 502, this force on one side of the device 502 causes the device to deflect away from the guidewire 560 in a direction corresponding the to the location at which the pull wire 514 is attached to the device. This direction may be shown by the arrow 592.

Persons skilled in the art will recognize that the apparatus, systems, and methods described above can be modified in various ways. Accordingly, persons of ordinary skill in the art will appreciate that the aspects encompassed by the present disclosure are not limited to the particular exemplary aspects described above. In that regard, although illustrative aspects have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
an endovascular catheter or guidewire configured to be positioned within a blood vessel of a patient, wherein the endovascular catheter of guidewire comprises:
a first set of electrodes comprising a first electrode and a second electrode; and
a second set of electrodes comprising a third electrode and a fourth electrode; and
a processor circuit configured for communication with the endovascular catheter or guidewire, wherein the processor circuit is configured to:
control the first set of electrodes such that an electrical current is transmitted between the first electrode and the second electrode;
control the second set of electrodes to obtain a first measurement of a voltage difference between the third electrode and the fourth electrode;
determine, based on the first measurement of the voltage difference, a first flow metric for blood flow within the blood vessel; and
output the first flow metric to a display in communication with the processor circuit.

2. The system of claim 1, further comprising a power source configured for electrical communication with the processor circuit and the first set of electrodes.

3. The system of claim 2, wherein, to control the first set of electrodes, the processor circuit is configured to control the power source to provide the electrical current to only the first electrode.

4. The system of claim 3, wherein the first electrode is configured to transmit the electrical current to the second electrode using a first electrical circuit formed between the first electrode and the second electrode by the blood flow.

5. The system of claim 4, wherein the second set of electrodes is configured to obtain the first measurement of the voltage difference using a second electrical circuit formed between the third electrode and the fourth electrode by the blood flow.

6. The system of claim 4, wherein the electrical current comprises a constant electrical current.

7. The system of claim 1, further comprising at least one of an amplifier or a rectifier configured for electrical communication with the second set of electrodes.

8. The system of claim 5, wherein the processor circuit is configured to control at least one of the amplifier or the rectifier to determine the first measurement of the voltage difference based on the electrical communication with the third electrode and the fourth electrode.

9. The system of claim 1, wherein the third electrode and the fourth electrode are positioned between the first electrode and the second electrode.

10. The system of claim 1,
wherein the first electrode and the second electrode comprise a first spacing, and
wherein the first electrode and the third electrode comprise a second spacing less than the first spacing.

11. The system of claim 1, wherein the blood vessel comprises a renal artery.

12. The system of claim 1, further configured to:
control the second set of electrodes to obtain a second measurement of the voltage difference between the third electrode and the fourth electrode; and
determine, based on the first measurement of the voltage difference, a second flow metric for the blood flow.

13. The system of claim 12, wherein the processor circuit is configured to output the second flow metric to the display.

14. The system of claim 12, wherein the processor circuit is configured to compare the first flow metric and the second flow metric.

15. The system of claim 14, wherein the processor circuit is configured to provide an output based on the comparison to the display.

16. The system of claim 14,
wherein the blood vessel is a renal artery,
wherein the first measurement is obtained before a stimulation of a sympathetic nervous system of the patient and the second measurement is obtained after the stimulation, and
wherein the processor circuit is configured to:
determine whether renal denervation is recommended for the patient based on the comparison; and
provide an output based on the determination to the display.

17. The system of claim 14,
wherein the blood vessel is a renal artery,
wherein the first measurement is obtained before a renal denervation procedure and the second measurement is obtained after the renal denervation procedure, and
wherein the processor circuit is configured to:
determine if the renal denervation procedure was successful based on the comparison, and
provide an output based on the determination to the display.

18. A system, comprising:
an endovascular catheter or guidewire configured to be positioned within a renal artery of a patient, wherein the endovascular catheter of guidewire comprises:
an outer set of electrodes comprising a first electrode and a second electrode; and
an inner set of electrodes comprising a third electrode and a fourth electrode; and
a processor circuit configured for communication with a power source and the endovascular catheter or guidewire, wherein the processor circuit is configured to:
control the power source to provide constant electrical current to the first electrode such that the constant electrical current is transmitted from the first electrode to the second electrode with a first electrical circuit formed between the first electrode and the second electrode by blood flow within the renal artery;
control the third electrode and the fourth electrode to obtain a measurement of a voltage difference with a second electrical circuit formed between the third electrode and the fourth electrode with the blood flow, wherein the voltage difference is generated based on a change in the blood flow;
determine, based on the measurement of the voltage difference, a flow metric representative of the change in the blood flow; and
provide an output based on the flow metric to a display in communication with the processor circuit.
